(19) United States
(12) Patent Application Publication
(10) Pub. No.: US 2015/0088022 A1
(43) Pub. Date: Mar. 26, 2015

(54) SYSTEMS AND METHODS FOR REMOTE MONITORING OF PATIENT TREATMENT

(71) Applicants: WILLIAM B. CRIBBS, MORRO BAY, CA (US); ARNOLD RUGAMA, FRESNO, CA (US)

(72) Inventors: WILLIAM B. CRIBBS, MORRO BAY, CA (US); ARNOLD RUGAMA, FRESNO, CA (US)

(21) Appl. No.: 14/036,277

(22) Filed: Sep. 25, 2013

(51) Int. Cl. A61B 5/00; G06Q 50/22; A61B 5/08

(52) U.S. Cl. CPC A61B 5/0022; A61B 5/08; G06Q 50/22
USPC 600/529; 705/2

(57) ABSTRACT

Systems and methods for remote monitoring of patient treatment are disclosed. A medical device may transmit telemetry directly to a patient terminal, or the medical device may be retrofitted with instrumentation for the patient terminal to collect telemetry. The patient terminal may collect patient self-reporting data. The telemetry and the self-reporting data may be sent to a cloud server, which may store the patient data. The patient data may be used to generate an alert, or it may be sent to a doctor terminal for display and review.

### BACKGROUND

The embodiments herein relate generally to systems and methods for the remote monitoring of patient treatment, including gathering medical device telemetry and patient self-assessment data.

Currently, a physician is not able to accurately and regularly monitor a patient while the patient is at home. Current methods of home monitoring, including, e.g., calling, device logs, office visits, and so on, do not provide effective, timely, or efficient information to the responsible physician. Inaccurate or incomplete monitoring presents challenges to the physician in terms of making a diagnosis, determining treatment effectiveness, determining patient compliance, completing medical paperwork, and other areas.

Some current medical devices provide some telemetry, such as when devices are used by patients during sleep. However, current telemetry is not only limited in scope, it is also limited to being integrated in new products (retrofitting or backwards compatibility is not possible) and operating on proprietary protocols and methods. Proprietary methods “lock” medical professionals into a problematic situation where only one kind of bedside device can be used and other devices are rendered inoperable. Standard compliance telemetry is insufficient for proper evaluation of treatment effectiveness. In some extreme cases, without physician personal intervention, even with “passing” compliance telemetry, the treatment is proven to be completely ineffective to the detriment of the patient. Furthermore, current systems do not allow patient self-reporting, which is essential for evaluating effectiveness of treatment.

### SUMMARY

According to one embodiment of the present invention, a remote patient feedback system is configured to electronically transmit real-time patient data about a patient undergoing treatment. The system is further configured to receive objective physiological patient data and subjective patient self-reporting data and to combine the objective data and the subjective data for transmission to a health care professional. The system includes a sensor and a computer system. The sensor is configured to detect an objective physiological parameter of a patient in real-time. The computer system is configured to process and store object patient data and subjective patient data, the objective patient data comprising the objective physiological parameter detected by the sensor. The computer system includes a display device, an electronic interface, a memory device, and a network device. The display device is configured to display a graphical user interface comprising an interactive option configured to permit input of subjective patient self-reporting data based upon at least one of how the patient is responding to treatment and how the patient is feeling at any one point in time. The subjective patient self-reporting data enhances the value of the objective physiological data when both are transmitted to the patient's health care professional. The electronic interface is configured to connect to the sensor and to receive objective patient data from the sensor. The memory device is configured to store objective and subjective patient data. The network device is configured to connect to a remote computer system and to digitally transmit the objective and subjective patient data to the patient's health care professional.

In addition, the system may include a medical device that includes the sensor. The medical device may include a continuous positive airway pressure (CPAP) machine. The sensor may include a pressure sensor configured to be connected to a breathing tube used by a patient without disrupting a flow of air to and from the patient.

According to an additional embodiment of the present invention, a computerized method stores and transmits patient data, including objective patient physiological data and subjective patient self-reporting data. The method includes store a health care professional profile associated with a health care professional. The method includes storing a patient profile associated with a patient and associating the patient profile with the health care professional profile. The method includes receiving from a remote computer system and storing objective physiological data and subjective patient self-reporting data. The objective patient data includes an objective physiological parameter of the patient. The subjective patient data includes an indication of how the patient is responding to treatment, an indication of how the patient is feeling at any one point in time, or both. The subjective data enhances the value of the objective physiological patient data when both are reviewed by the health care professional. The method includes transmitting the patient data to a doctor terminal associated with the health care professional.

In addition, the doctor terminal may include at least one of a website, a mobile app, a mobile device, a smartphone, a table, and a laptop. The method may further include storing an alter template that includes an alter criterion. The alert template may be associated with the doctor profile. The method may include comparing the patient data to the alert template, generating an alert if an alert criterion is satisfied, and transmitting the alert to the doctor terminal.

### BRIEF DESCRIPTION OF THE FIGURES

The detailed description of some embodiments of the present invention is made below with reference to the accompanying figures, wherein like numerals represent corresponding parts of the figures.

FIG. 1 shows a schematic view of medical device telemetry collection, according to one embodiment of the present invention.

FIGS. 2A and 2B show a flowchart of one embodiment of the present invention.

FIG. 3 shows a schematic view of an exemplary patient terminal, according to one embodiment of the present invention.

FIG. 4 shows a schematic view of an exemplary doctor terminal, according to one embodiment of the present invention.

### DETAILED DESCRIPTION OF CERTAIN EMBODIMENTS

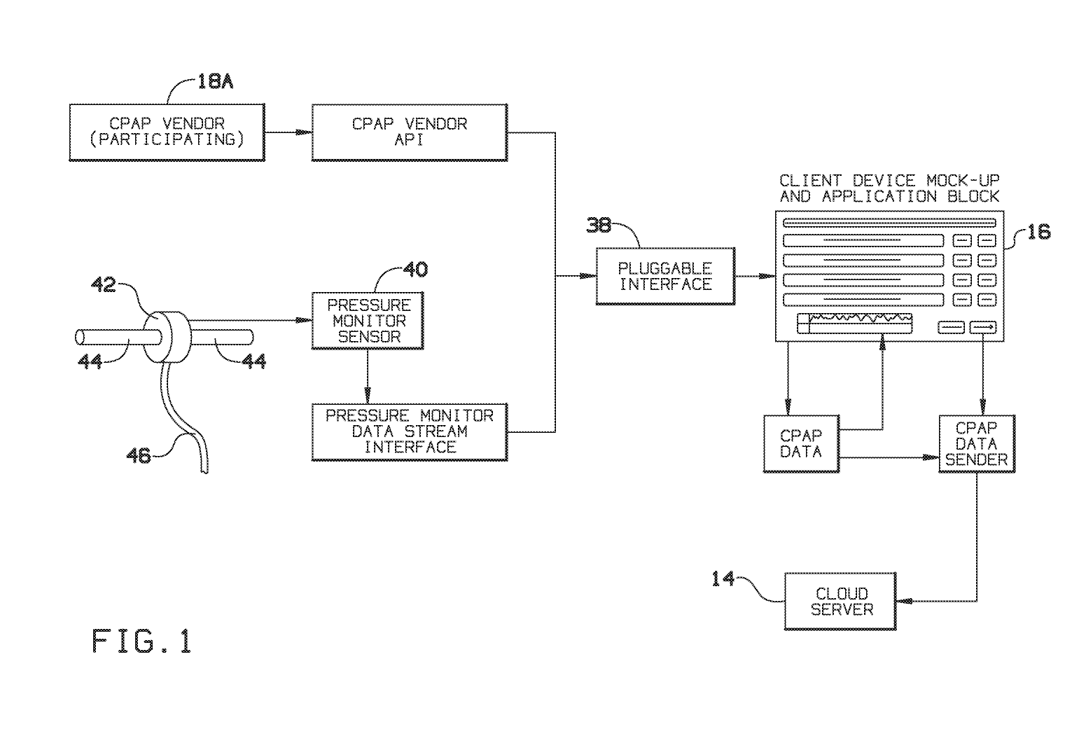

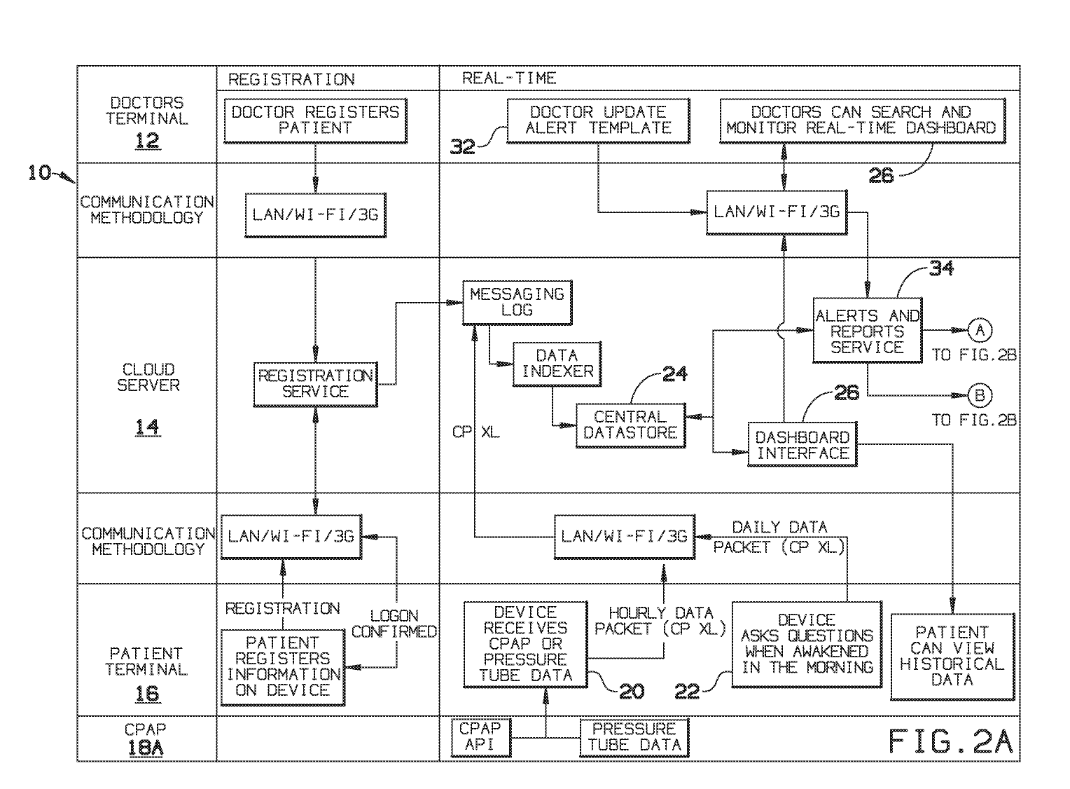

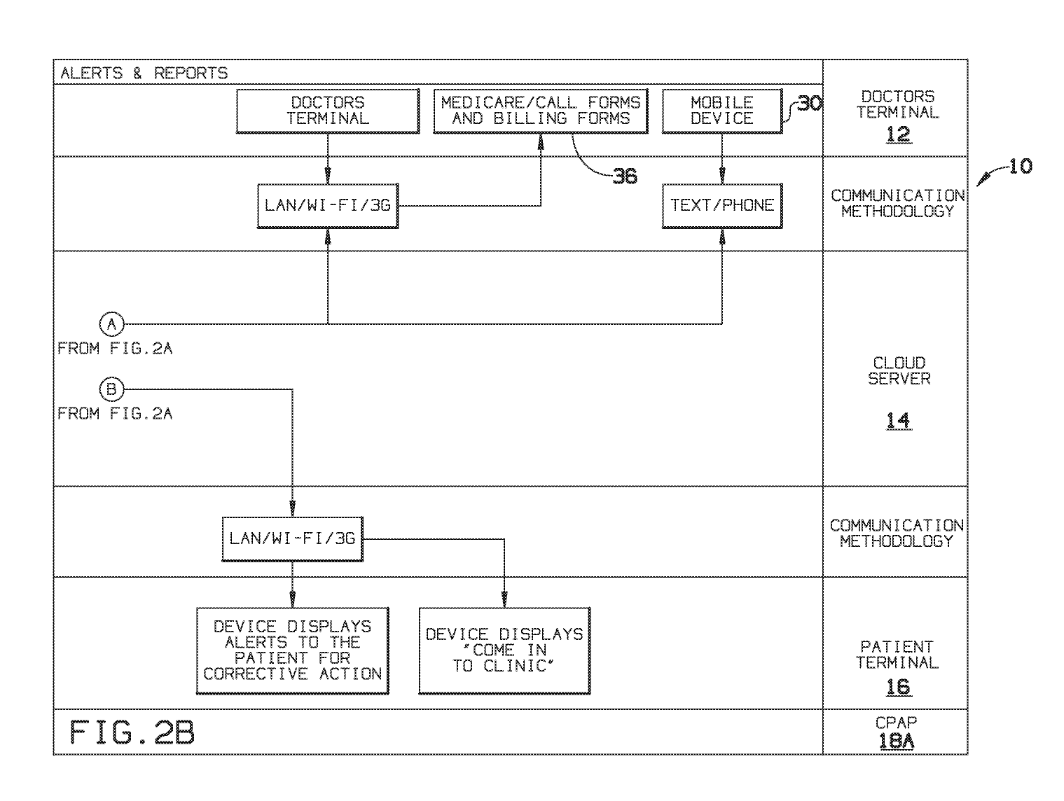

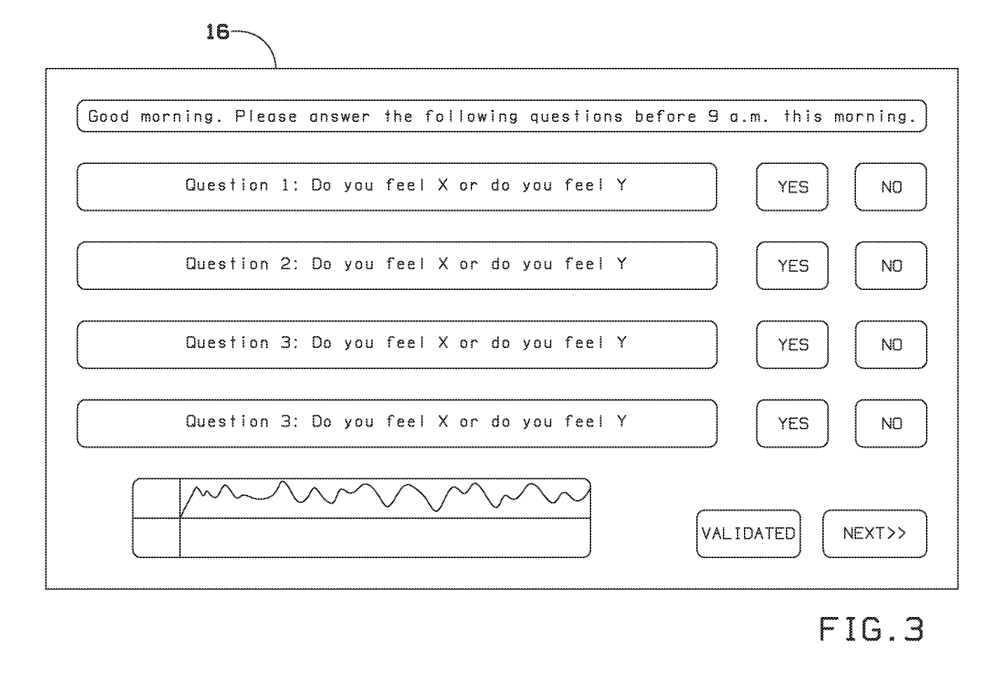

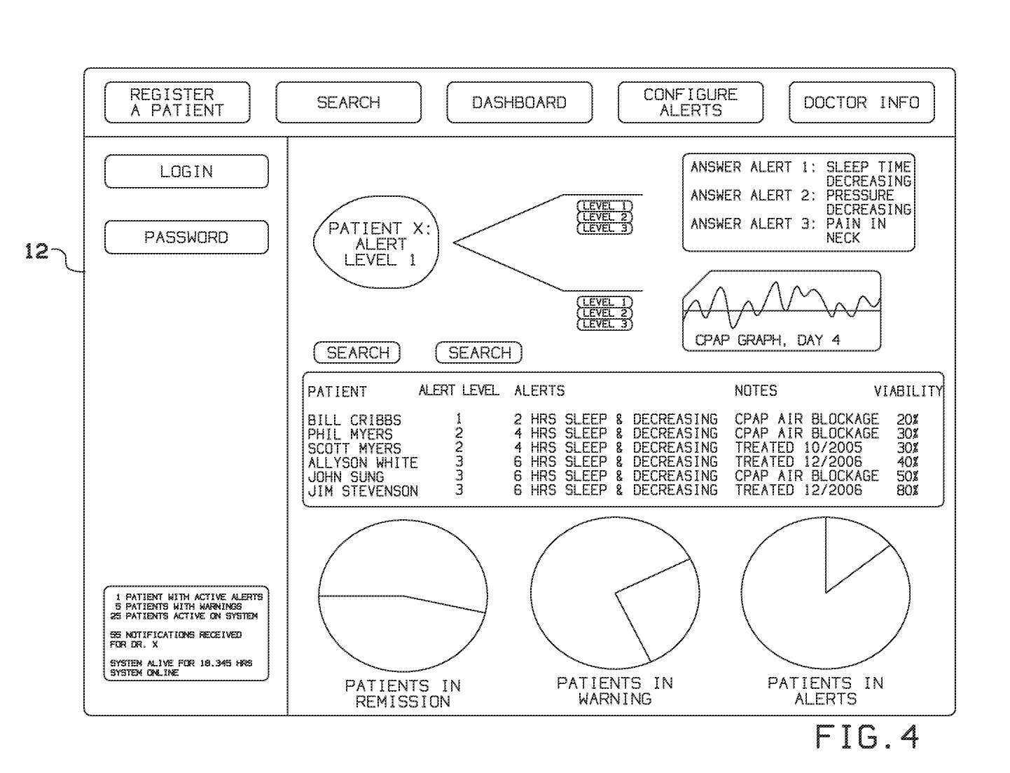

By way of example, and referring to FIGS. 1-4, one embodiment of the present invention comprises a system 10 for monitoring patient treatment. The system 10 may include a doctor terminal 12, a cloud server 14, a patient terminal 16, and a medical device 18, such as, e.g., a continuous positive airway pressure (CPAP) machine 18A. The system 10 may use telemetry data logging and client-server information technology to provide data streams from the patient terminal 16 to the cloud server 14 for exploitation. For example, patient terminal 16 may be situated in a patient's home, where it may be connected to, e.g., a CPAP machine 18A or other medical device 18. While the patient is sleeping, the data 20 from the CPAP 18A may be uploaded from the patient terminal 16 to the cloud server 14 over an existing or proprietary network. Referring to FIG. 4, when the patient wakes up, the patient terminal 16 may ask pre-selected questions of the patient, in a process known as patient self-reporting 22. The patient terminal 16 may upload the self-reporting data 22 to the cloud server 14. The cloud server 14 may store and/or index the CPAP data 20, the self-reporting data 22, or both in a database or other facility 24.

The doctor terminal 12 may accesses the cloud server 14 via an existing or proprietary network. The doctor terminal 12 may include, e.g., a dashboard website 26 or web application running on a personal computer, a mobile app running on a smartphone or tablet 30, and so on. The doctor terminal 12 may provide efficient, timely access to treatment data. A doctor using the doctor terminal 12 may search patient data, configuring self-reporting questions, and create reports from patient data. A doctor or other provider may use the doctor terminal 12 to enter an alert template 32 into the cloud server 14, and the cloud server 14 may use data analysis 34 and alert templates 32 to create one or more alerts. An alert may manifest as a notification to doctor, patient, or both, prompting additional physician action or treatment modification. The data logging feature 24 of the cloud sever 14 may provide the ability to create reports or automatically create or export data to medical billing forms 36.

The patient terminal 16 may include a standalone device, which may include a display, an input device, memory, a processor, a connector for connecting to the medical device 18, and a communication module. The communication module may include, for example, a modem, a wireless networking device, a mobile wireless radio, or the like. The patient terminal 16 may include an application that runs on a mobile device platform, such as, e.g., a smartphone, tablet, or the like. The patient terminal 16 may include short-range wired or wireless communication interface 38, such as, e.g., USB, NFC, Bluetooth, or the like, for communicating with a medical device 18 or medical device sensor 40. For example, the medical device 18 may include a CPAP 18A that is configured to communicate with the patient terminal 16 via communication interface 38. To work with a CPAP 18A that lacks this capability, the patient terminal may include an airway connector 42 that may join two sections of airway tubing 44. A pressure tube 46 may be attached to the connector 42. The connector 42 may include a device sensor 40, such as, e.g., a pressure sensor. Other types and arrangements of sensors are contemplated for use with different medical devices 18. The connector 42 may be configured to connect to the communication interface 38.

The cloud server 14 may parse incoming messages and store data in a database 24. The messages may include data that has been formatted according to an XML protocol. For example, an open XML standard or protocol may be used to allow medical devices 18 to communicate with the server 14. The protocol or message standard may include, e.g., patient information, device information, daily device record, patient self-reporting data, device history, or a combination thereof The patient information may include, e.g., name, address, unique identifier, geolocation, and/or device identity. The device information may include, e.g., an identification string. The daily device record may include, e.g., one or more readings from the medical device 18. Each reading may include, e.g., date and time of the reading, and the reading itself For example, the reading itself may include a pressure reading for a CPAP 18A. The patient self-reporting data may include, e.g., one or more doctor's questions and the patient's answer to each question. The device history may include, e.g., some or all device readings from the current time to a past time, and the history may include all readings stored on the medical device 18 or patient terminal 16.

Persons of ordinary skill in the art may appreciate that numerous design configurations may be possible to enjoy the functional benefits of the inventive systems. Thus, given the wide variety of configurations and arrangements of embodiments of the present invention the scope of the present invention is reflected by the breadth of the claims below rather than narrowed by the embodiments described above.

What is claimed is:

1. A remote patient feedback system configured to electronically transmit real-time patient data about a patient undergoing treatment, the system configured to receive objective physiological patient data and subjective patient self-reporting data and to combine the objective data and the subjective data for transmission to a health care professional, the system comprising:
a sensor configured to detect an objective physiological parameter of a patient in real-time; and
a computer system configured to process and store objective patient data and subjective patient data, the objective patient data comprising the objective physiological parameter detected by the sensor, the computer system comprising:
a display device configured to display a graphical user interface comprising an interactive option configured to permit input of subjective patient self-reporting data based upon at least one of how the patient is responding to treatment and how the patient is feeling at any one point in time, the subjective patient data enhancing the value of the objective physiological patient data when both are transmitted to the patient's health care professional,
an electronic interface configured to connect to the sensor and to receive object patient data from the sensor,
a memory device configured to store objective and subjective patient data, and
a network device configured to connect to a remote computer system and to digitally transmit the objective and subjective patient data to the patient's health care professional.

2. The system of claim 1, further comprising:
a medical device comprising the sensor.

3. The system of claim 2, wherein the medical device comprises a continuous positive airway pressure (CPAP) machine.

4. The system of claim 1, wherein the sensor comprises a pressure sensor configured to be connected to a breathing tube used by the patient without disrupting a flow of air to and from the patient.

5. A computerized method for storing and transmitting patient data, including objective patient physiological data and subjective patient self-reporting data, the method comprising:
storing a health care professional profile associated with a health care professional;
storing a patient profile associated with a patient and associating the patient profile with the health care professional profile;
receiving from a remote computer system and storing objective patient physiological data and subjective patient self-reporting data, the objective patient data comprising an objective physiological parameter of the patient, the subjective patient data comprising at least one of an indication of how the patient is responding to treatment and an indication of how the patient is feeling at any one point in time, the subjective patient data enhancing the value of the objective physiological patient data when both are reviewed by the health care professional; and
transmitting the patient data to a doctor terminal associated with the health care professional.

6. The method of claim 5, wherein the doctor terminal comprises at least one of a website, a mobile app, a mobile device, a smartphone, a table, and a laptop.

7. The method of claim 5, further comprising:
storing an alert template comprising an alert criterion, the alert template associated with the doctor profile;
comparing the patient data with the alert template;
generating an alert if an alert criterion is satisfied; and
transmitting the alert to the doctor terminal.
